(19) United States
(12) Patent Application Publication
(10) Pub. No.: US 2023/0380777 A1
(43) Pub. Date: Nov. 30, 2023

(54) IMPROVED DOSING METHOD FOR POSITRON EMISSION TOMOGRAPHY IMAGING

(71) Applicant: Ottawa Heart Institute Research Corporation, Ottawa (CA)

(72) Inventor: Robert A. DeKemp, Ottawa (CA)

(73) Assignee: Ottawa Heart Institute Research Corporation, Ottawa (CA)

(21) Appl. No.: 18/320,115

(22) Filed: May 18, 2023

Related U.S. Application Data
(63) Continuation-in-part of application No. PCT/IB2022/053707, filed on Apr. 20, 2022.
(60) Provisional application No. 63/226,947, filed on Jul. 29, 2021.

(51) Int. Cl. A61B 6/03; A61M 5/168; G16H 20/40; A61B 6/00

(52) U.S. Cl. CPC A61B 6/037; A61M 5/168; A61M 2205/50; A61B 6/481; G16H 20/40

(57) ABSTRACT

The present invention provides an improved method of PET imaging in a subject for diagnosis and/or treatment of cardiovascular related disease. The method comprises exponential function dosing based on subject body habitus. It provides improved and consistent imaging quality in comparison to fixed or linear dosing based on subject's body weight. More particularly, it relates to a method of imaging processing for diagnosing and/or identifying a risk of developing a coronary artery disease comprising administering a dose of Rb-82 to a subject, wherein the dose is calculated based on exponential squared function of body habitus of the subject; and wherein the method of imaging processing in a subject is iterative ordered-subset expectation maximization (OSEM) reconstruction method.

### TECHNICAL FIELD

The present invention relates in general to nuclear imaging and medicine, in particular, to Positron Emitting Tomography (PET) for diagnosing and/or treating a disease or a condition.

### BACKGROUND

Radioisotopes play a pivotal role in diagnosis and mitigation of various diseased conditions. For example, ⁶⁰Co in treatment of cancer, ¹³¹I in treatment of hyperthyroidism, ¹⁴C in breath tests, ^{99m}Tc and ⁸²Rb as tracers in myocardial perfusion imaging. The radioisotopes for pharmaceutical use are produced either by nuclear bombardment in cyclotron in specially approved remote sites or in-situ by employing radioisotope generators at the site of use.

Rubidium (⁸²Rb) is used as a positron emission tomography (PET) tracer for non-invasive measurement of myocardial perfusion. Rubidium-82 is produced in-situ by radioactive decay of strontium-82. Rubidium elution systems utilize doses of rubidium-82 generated by elution within a radioisotope generator, and infuse the radioactive solution into a patient. The infused dose of radiopharmaceutical is absorbed by cells of a target organ of the patient and emit radiation, which is detected by a PET scanner in order to generate an image of the organ.

Selecting an appropriate imaging protocol and administered activity appropriate for each patient's body habitus is important to obtain diagnostic image quality in every study (Henzlova M J, Duvall W L, Einstein A J, Travin M I, Verberne H J. ASNC imaging guidelines for SPECT nuclear cardiology procedures: Stress, protocols, and tracers. J Nucl Cardiol. 2016; 23(3):606-39). Imaging with PET is susceptible to the patient's body habitus, as the increase in body weight leads to higher fractions of attenuated and scattered photons resulting in lower quality images. Applying methods such as time-of-flight (TOF) image reconstruction and body weight-based tracer dosing or image smoothing can help to reduce noise and improve image quality. Historically, Rb-82 PET imaging was performed using a single fixed dose for all patients, due in part to limitations of early-generation tracer delivery systems, which is known to result in lower count-density and image quality in larger patients. This undesirable effect of old PET imaging system can be mitigated to some extent by administration of activity as a linear function of body weight using the advanced and latest generation Rb-82 elution system (Ruby-Fill)-USFDA approved rubidium elution system for myocardial perfusion imaging, marketed by Jubilant Radiopharma. The present inventor observed that the linear weight based dosing recommended by Van Dijk et al (Journal of Nuclear Cardiology, 2019) does not provide consistent image quality over a broad range of body habitus.

Although the European Association of Nuclear Medicine (EANM) guidelines still accept the use of fixed dosing for Rb-82 ranging from 740 to 1110 MBq depending on the PET-CT device sensitivity, the recommended tracer dosing for Rb-82 PET imaging in 3D-mode is 10 MBq/kg (with a minimal dose of 740 MBq and maximal dose of 1480 MBq). However, this weight-based dosing approach as a linear function of patient weight does not necessarily result in uniform image quality across all patients with variant body habitus. Furthermore, the lower limit of 740 MBq may not allow adequate dose reduction in very small or pediatric patients, and conversely, the upper limit of 1480 MBq may not allow adequate image quality in the largest patients. In one study, Masuda et al, Comparison of imaging protocols for 18F-FDG PET/CT in overweight patients: Optimizing scan duration versus administered dose. J Nucl Med, June 2009, 50 (6) 844-848) showed that increasing the dose linearly per kilogram of body weight did not improve the quality of PET-CT images. Moreover, radiopharmaceuticals dosing is a critical part of any successful imaging technique as lower or higher dosing in a subject may cause unnecessary radiation exposure to subjects that may be hazardous. Saline flush is one of the techniques, wherein saline is pushed into the subject to deliver the remaining activity of imaging agent into the subject to improve the imaging quality, however, it is also not found to provide consistent image quality over a wide range of patient weight. Significantly, a shorter half-life of seconds of Rubidium-82 in comparison to other radionuclides and variation of cardiac image quality with changes in myocardial blood flow with coronary disease, cardiac output are key challenges for implementing exponential function based dosing for PET imaging of the heart.

Selecting an appropriate imaging protocol including administered activity appropriate for each patient's body habitus is very important to optimize diagnostic image quality. Current SPECT imaging guidelines from the American Society of Nuclear Cardiology (ASNC) suggest “ . . . an effort to tailor the administered activity to the patient's habitus and imaging equipment should be made . . . [however] strong evidence supporting one particular weight-based dosing scheme does not exist.” Similarly for PET, the current ASNC perfusion imaging guidelines suggest that “Large patients may benefit from higher doses” but no specific recommendations are provided to ensure consistent image quality for ⁸²Rb MPI.

Image smoothing can help to reduce noise and improve image quality, but at the expense of lower spatial resolution. Alternatively, longer scanning times and/or weight-based tracer dosing have been proposed and are currently recommended as a solution to help standardize image quality in whole-body oncology PET imaging with F-18-fluorodeoxyglucose (¹⁸FDG). Historically, ⁸²Rb PET imaging has been performed using a single constant dose for all patients due in part to limitations of early generator systems which were calibrated for dose delivery at a single activity value but this is known to result in lower count-density and corresponding lower image quality in larger patients. The inventors have shown previously that this variation of image quality can be mitigated to some degree by the administration of activity in proportion to body weight using a new generation Rb-82 elution system. Contrary to ¹⁸FDG PET imaging, longer scan times cannot be used to improve ⁸²Rb image quality in these patients due to the ultra-short half-life of 75 seconds.

In earlier methods, the effects of proportional dosing to produce constant LV_{MEAN} activity values in the heart are partially consistent in the ⁸²Rb PET study and it is reported that the number of recorded ‘net’ coincidences (prompts-randoms) was constant over a wide range of patient weights. However, unlike the prior art study, which found no differences in body weight among the different categories of visual image quality with proportional dosing, the study results of the present invention demonstrate statistically significant decreases in image quality (assessed visually and quantitatively) as a function of body weight with proportional dosing. The pattern of decreasing image quality (despite constant tissue activity and ‘net’ coincidence counts) is likely due to the degrading effects of tissue attenuation on image quality. The experimental results of the present invention suggest that the increasing noise effects of PET attenuation are approximately linear with patient weight, and these can be corrected with the exponential dosing protocol, to produce organ activity values that increase linearly with weight. In contrast, the inventors of the present invention found improved standardization using exponential dosing with rubidium PET. While image quality is affected by the Poisson distribution of counting statistics, the noise effects and correction methods for the physical effects of scatter and attenuation (as well as random and prompt-gamma coincidences in PET) are quite different, which may explain the different results in SPECT vs PET.

The European Association of Nuclear Medicine (EANM) guidelines for PET MPI currently recommends weight-based tracer dosing for Rb-82 imaging in 3D-mode at 10 MBq/kg (with a minimal dose of 740 MBq and maximal dose of 1480 MBq) whereas the ASNC PET MPI guidelines still accept the use of a single constant dose of ⁸²Rb ranging from 740 to 1110 MBq depending on the PET-CT device sensitivity. The common lower limit of 740 MBq may not allow adequate dose reduction in very small or pediatric patients, whereas the upper limit of 1110 to 1480 MBq may not allow adequate image quality in the largest patients.

The results of the present invention have important implications for pediatric imaging studies such as Kawasaki Disease where PET imaging has been used to guide clinical management. In pediatric patients, the effective dose constant (radiation risk) is typically higher per unit activity injected (e.g. 4.9 vs 1.1 mSv/GBq in a 5-year-old vs adult patient) reflecting the higher organ activity concentrations and smaller distances between organs. The present invention suggests that the injected activity (and radiation effective dose) can be substantially reduced in the smallest patients while still maintaining diagnostic image quality.

The inventors have used weight-based dosing as a proportional function of patient weight (9-10 MBq/kg) to reduce variations of image quality depending on body habitus, and to reduce detector saturation during the tracer first-pass for accurate blood flow quantification. Despite this approach, larger patients still suffer from reduced 82 Rb PET image quality which is not aligned with the recommended principles of patient-centered imaging. Administration of Rb-82 activity as a fixed constant dose or in proportion to weight, results in stress PET perfusion image quality that decreases with patient weight. Exponential dosing as a squared function of patient weight (0.1 MBq/kg²) was found to standardize ECG-gated image quality across a wide range of weights, consistent with the goals of high-quality and patient-centered imaging. The proposed protocol and dosing method of the present invention can distribute the population dose from the smaller towards the larger patients as needed to maintain image quality, without increasing the average dose.

Hence, there exists an unmet urgent need to investigate and implement exponential dosing based on subject habitus or infusion parameters for PET or SPECT imaging in a subject. Optimal dosing maintains image quality in larger patients and lowers radioactivity dose in smaller patients compared to uniform/fixed or simple linear-weight-based dosing.

### SUMMARY

The present invention aims to provide a novel PET or SPECT imaging approach.

It is an object of the present invention to provide optimal dosing for maintaining a consistent image quality irrespective of subject body habitus.

It is an object of the present disclosure to provide dosing based on exponential function of subject body habitus for PET or SPECT imaging.

It is an object of the present disclosure to provide exponential dosing based on subject body habitus comprising body weight, body height, body surface area, lean body mass, body mass index, thoracic, and abdominal circumference or combinations thereof for PET or SPECT imaging.

It is an object of the present disclosure to provide radionuclide dosing based on exponential function of subject body habitus, wherein body habitus comprises body weight, body height, body surface area, lean body mass, body mass index, thoracic or abdominal circumference or other similar measures for Rb-82 PET imaging, diagnosis and/or treatment of heart related disorders.

It is an object of the present disclosure to provide radionuclide dosing based on exponential function of radionuclide generation and/or infusion system parameters comprising infusion time, infusion rate, type of radionuclide, dosing, parent isotope breakthrough, activity detector calibration and radionuclide generator age or combinations thereof.

It is an object of the present disclosure to provide radionuclide dosing based on exponential function of imaging system parameters comprising imaging scanner sensitivity, and imaging scanner/camera resolution or combination thereof.

It is an object of the present disclosure to provide exponential function of the body habitus based dosing for Rb-82 PET imaging.

It is an object of the present disclosure to provide consistent left ventricle (LV) myocardium signal-to-noise ratio (SNR) and myocardium-to-blood contrast-to-noise ratio (CNR) values across a wide range of patient body sizes when using exponential dosing of Rb-82 in comparison to a matched group of patients with linear dosing or fixed dosing.

It is an object of the present disclosure to provide accurate Rb-82 injected activity over the full range of injected doses prescribed from 0.01 to 10,000 MBq.

It is an object of the present disclosure to provide exponential function of the body habitus based dosing for Rb-82 PET imaging for diagnosing a subject suffering from or at a risk of developing coronary artery disease, ischemic and non-ischemic heart disease, and other organ diseases such as liver, kidney, spleen, adrenal, pancreas, brain or combinations thereof.

It is an object of the present disclosure to provide exponential function of the patient weight-based dosing for PET imaging.

It is an object of the present disclosure to provide standardized image quality across a wide range of body habitus.

It is an object of the present disclosure to provide standardized image quality across a wide range of subject weight, height, body mass and surface area or combinations thereof.

It is an object of the present disclosure to provide consistent signal-to-noise ratio (SNR) and contrast-to-noise ratio (CNR) across a wide range of subject body habitus.

It is an object of the present disclosure to provide improved image quality independent of variation is subject body habitus.

It is an object of the present disclosure to provide consistent image quality for administered radionuclide activity in the range from 0.01 to 10,000 MBq.

It is an object of the present disclosure to provide consistent dosing via automated generation and/or infusion system.

The present invention concerns any of the following items:

In one aspect of the present invention, a method of imaging a subject for diagnosing and/or identifying a risk of developing a coronary artery disease comprises administering a dose of Rb-82 to a subject, wherein the dose is calculated based on exponential function of body habitus of the subject.

In another aspect of the present invention, the body habitus comprises body weight, body height, body surface area, lean body mass, body mass index, and thoracic or abdominal circumference or combinations thereof.

In another aspect of the present invention, the dose can be further adjusted based on additional parameters selected from left ventricle ejection fraction, infusion time, infusion rate, imaging scanner sensitivity, type of radionuclide, imaging scanner/camera resolution and radionuclide generator age, generator yield or combination thereof.

In another aspect of the present invention, the imaging agent or radionuclide is generated and administered by automated infusion system.

In another aspect of the present invention, the automated radioisotope generation and infusion system comprises Rb-82 elution system.

In another aspect of the present invention, the dose is based on exponential function of the subject weight or subject height.

In another aspect of the present invention, one example of exponential function based dosing is calculated by activity=0.1×weight, wherein the body weight is in kilograms and activity is in MBq.

In another aspect of the present invention, consistent image quality observed in the dose range of 1 MBq to 10,000 MBq.

In another aspect of the present invention, the method further comprises administering a stress agent to the subject. In another aspect of the present invention, the method comprises inducing stress to the subject.

In another aspect of the present invention, the stress can be induced by exercise or administering a stress agent selected from adenosine, adenosine triphosphate, regadenoson, dobutamine, and dipyridamole.

In another aspect of the present invention, the imaging comprises PET or SPECT imaging.

In another aspect of the present invention, the subject's weight ranges from 1 kg to 300 kg.

In another aspect of the present invention, the dose of the imaging agent to be administered is calculated by automated generation and infusion system.

In another aspect of the present invention, a method of obtaining Rb-82 PET images of the region of interest of the subject with consistent image quality, wherein the dose of imaging agent is calculated based on exponential function of subject body habitus.

In another aspect of the present invention, the image quality is independent of body habitus variation in the subjects.

In another aspect of the present invention, the consistency of image quality is measured by coefficient of variation of signal to noise ratio and/or contrast to noise ratio measured over a subject weight range of 10 to 200 kg for exponential weight based dosing and linear weight based dosing.

In another aspect of the present invention, the coefficient of variation for exponential weight based dosing ranges from about 15 to 30 percent.

In another aspect of the present invention, the coefficient of variation for exponential weight based dosing is less than about 30 percent, preferably less than about 20 percent, more preferably less than about 15 percent.

In another aspect of the present invention, the coefficient of variation for exponential weight based dosing ranges from about 12 to 26 percent.

In another aspect of the present invention, a method of imaging a subject suffering from or at a risk of developing a coronary artery disease comprising: calculating the dose based on exponential function of body habitus; generating a calculated dose of Rb-82 by automated elution system; administering the generated dose of Rb-82 to the subject; performing PET imaging to obtain images; optionally, administering the dose of stress agent and performing PET imaging to obtain images; and performing an assessment of the obtained images for diagnosis and/or treatment of the suspected disease.

### BRIEF SUMMARY OF DRAWINGS

Further features and advantages of the present invention will become apparent from the following detailed description, taken in combination with the appended drawings, in which:

FIG. 1 Depicts a diagram schematically demonstrating principal elements of an automated Rb-82 generation and infusion system for patient in accordance with an embodiment of the present invention.

FIG. 2 Depicts a block diagram schematically demonstrating key elements of an automated Rb-82 generation and infusion system quality control test with dose calibrator in accordance with another embodiment of the present invention.

FIGS. 3A and 3B depict Rb-82 PET images in a 35 kg patient (left) and 180 kg patient (right) acquired with proportional dosing following linear weight-based administration of approximately 5-20 MBq/kg tracer activity. Lower image quality is observed in the larger patient. FIGS. 3C and 3D are Rb-82 PET static (ungated) images acquired with exponential dosing and the image quality is observed similar between larger and smaller patients.

FIGS. 4A-4D depict SNR and CNR functions of patient weight in the linear and exponential dosing groups, for ECG-gated (FIG. 4A, FIG. 4B) and ungated static (FIG. 4C, FIG. 4D) rubidium-82 PET images acquired during dipyridamole stress.

FIGS. 5A and 5B depict measured power function exponent (Beta) values and 95% confidence intervals showing the dependence of image quality (SNR and CNR) on patient weight for the ECG-gated (FIG. 5A) and ungated static (FIG. 5B) PET images.

FIG. 6 Depicts Regions-of-interest (ROI) drawn in the heart (A) for measurement of SNR and CNR. LV_{MAX} was taken within the three-dimensional region of the myocardial wall (white) identified automatically by the Corridor-4DM software. Blood mean and standard deviation were taken in a region drawn manually in the left atrial cavity (red) on a vertical long axis (V_{LA}) image.

FIG. 7 Depicts Bland-Altman plots of inter-operator reproducibility for the measurements of heart image quality on static (A) and gated (B) images using proportional dosing and exponential dosing (C, D). Most of the variability in signal-to-noise ratio (SNR) and contrast-to-noise ratio (CNR) comes from the standard deviation (stdev) measurement in the blood (BL) cavity.

FIG. 8 Depicts weight-dependence in the measurements of heart image quality on static (A) and gated (B) images using proportional dosing and exponential dosing (C, D). Proportional dosing results in relatively constant LV_{MAX} activity whereas exponential dosing produces LV_{MAX} that increases linearly with patient weight.

FIG. 9 Depicts patient weight distributions in the exponential and proportional dosing cohorts.

FIGS. 10A-10C depict Rb-82 PET activity values on ECG-gated imaging with proportional and exponential dosing. LV_{MAX} (FIG. 10A) values are constant with proportional dosing but increase linearly by weight with exponential dosing, (FIG. 10B) Blood_{MEAN} activity values are almost constant with proportional dosing but increase by weight with exponential dosing, and (FIG. 10C) Blood_{SD} activity remains very similar between dosing protocols.

FIG. 11 Depicts Rb-82 PET visual image quality score (IQS_{HEART}).

FIG. 12 Depicts Box-plots of patient weight according to visual image quality score (IQS) in the proportional (A) and exponential (B) dosing groups.

FIG. 13 Depicts Rb-82 PET static-ungated SA (top) and ECG-gated HLA & VLA (bottom) images acquired with proportional (A, B) and exponential (C, D) dosing.

FIG. 14 Depicts Rb-82 PET static (ungated) images acquired with proportional (A, B) and exponential (C, D) dosing.

FIG. 15 Depicts Rb-82 PET contrast-to-noise ratio (CNR_{HEART}) decreases with increasing patient body weight in the proportional dosing cohort but not in the exponential dosing cohort for both ECG-gated (A) and ungated static (B) images. (C) Box-plots of CNR_{HEART}.

FIG. 16 Depicts Rb-82 PET signal-to-noise ratio (SNR_{BLOOD}) decreases with increasing patient body weight in the proportional dosing cohort (A) and tended to increase in the exponential dosing cohort (B). (C) Box-plots of the SNR_{BLOOD} show the summary effects of dosing method on the patient groups as a whole.

FIG. 17 Depicts Rb-82 PET liver signal-to-noise ratio (SNR_{LIVER}) decreases with increasing patient body weight (W) in the proportional dosing cohort (A) but not in the exponential dosing cohort (B). (C) Box-plots of the SNR LIVER show the summary effects of dosing method on the patient groups as a whole.

### DETAILED DESCRIPTION

In one aspect, the invention relates to the use of ⁸²Rb dosing as an exponential (squared) function of weight to standardize PET MPI quality across a wide range of patient body sizes, following a similar protocol validated previously for whole-body ¹⁸FDG PET. There is currently a need to improve PET or SPECT imaging. The present invention is based on unexpected discovery that administering a dose of radionuclide to a subject based on exponential function of subject body habitus provides consistent image quality irrespective of variation in subject body habitus or infusion system related parameters or imaging system parameters. The present inventors unexpectedly found that exponential-based dosing provides a consistent signal to noise and contrast to noise ratios over a wide range of subject body habitus. The present invention can be more readily understood by reading the following detailed description of the invention and included embodiments.

As used herein, the term ‘imaging’ refers to techniques and processes used to create images of various parts of the human body for diagnostic and treatment purposes within digital health. X-ray radiography, Fluoroscopy, Magnetic resonance imaging (MRI), Computed Tomography (CT), Medical Ultrasonography or Ultrasound Endoscopy Elastography, Tactile imaging, Thermography Medical photography, and nuclear medicine functional imaging techniques e.g. positron emission tomography (PET) or Single-photon emission computed tomography (SPECT). Imaging seeks to reveal internal structures of the body, as well as to diagnose and treat disease.

As used herein, the term ‘Positron Emission Tomography’ (PET) refers to a functional imaging technique that uses radioactive substances known as radiotracers or radionuclides to visualize and measure changes in metabolic processes, and in other physiological activities including blood flow, regional chemical composition, and absorption. Different tracers can be used for various imaging purposes, depending on the target process within the body commonly used radionuclide isotopes for PET imaging include Rb-82 (Rubidium-82), 0-15 (Oxygen-15), F-18 (Fluorine-18), Ga-68 (Gallium-68), Cu-61 (Copper-61), C-11 (Carbon-11), N-13 (Ammonia-13), Co-55 (Cobalt-55), Zr-89 (Zirconium-89). The preferred radionuclide comprises Rb-82 having a half-life of 75 seconds.

As used herein, the term ‘SPECT’ refers to a Single-photon emission computed tomography is a nuclear medicine tomographic imaging technique using gamma rays and providing true 3D information. This information is typically presented as cross-sectional slices through the patient, but can be freely reformatted or manipulated as required. The technique requires delivery of a gamma-emitting radioisotope (a radionuclide) into the patient, normally through injection into the bloodstream. A marker radioisotope is generally attached to a specific ligand to create a radio ligand, whose properties bind it to certain types of tissues. This allows the combination of ligand and radiopharmaceutical to be carried and bound to a region of interest in the body, where the ligand concentration is assessed by a gamma camera. SPECT agents include ^{99m}Tc technetium-99m (^{99m}Tc)-sestamibi, and ^{99m}Tc-tetrofosmin), In-111, Ga-67, Tl-201 (Thallium-201).

As used herein, the term ‘diagnosis’ refers to the process of identifying a disease, condition, or injury from its signs and symptoms. A health history, physical exam, and tests, such as blood tests, imaging, scanning, and biopsies can be used to help make a diagnosis.

As used herein, the term ‘body habitus’ refers to subject body physique or body build. Body habitus can comprise subject weight, height, body mass, lean body mass, body mass index, body surface area, thoracic or abdominal circumference or other similar measures of the subject.

As used herein, the term ‘assessment’ refers to a qualitative or quantitative assessment of the blood perfusion in a body part or region of interest.

As used herein, the term ‘stress’ or ‘stress agent’ refers to agents used to generate stress in a patient or a subject during imaging procedure. The stress agents according to the present invention are selected from vasodilator agents for example adenosine, adenosine triphosphate and its mimetic, A2A adenosine receptor agonist for example regadenoson or adenosine reuptake inhibitor dipyridamole, other pharmacological agent to increase blood flow to the heart, like catecholamines (for example dobutamine, acetyl-choline, papaverine, ergonovine, etc.) or other external stimuli to increase blood flow to the heart such as cold-pressor, mental stress or physical exercise.

As used herein, the term ‘automated infusion system or radionuclide generation and/or infusion system or Rb-82 elution system’ refers to system for generation and/or infusion of a radionuclide or radiotracer and administration into a subject. The automated infusion system comprises a radioisotope generator, dose calibrator, computer, controller, display device, activity detector, cabinet, cart, waste bottle, sensors, shielding assembly, alarms or alerts mechanism, tubing, source vial, diluent or eluant, valves. The automated infusion system can be communicatively or electronically coupled to imaging system.

As used herein, the term ‘dose’ refers to the dose of radionuclide required to perform imaging in a subject. The dose of a radionuclide to be administered to the subject ranges from 0.01 MBq to 10,000 MBq.

As used herein, the term ‘coronary artery disease’ refers to a disease of major blood vessels. Cholesterol-containing deposits (plaques) in coronary arteries and inflammation are causes of coronary artery disease. The coronary arteries supply blood, oxygen and nutrients to heart. A buildup of plaque can narrow these arteries, decreasing blood flow to heart. Eventually, the reduced blood flow may cause chest pain (angina), shortness of breath, or other coronary artery disease signs and symptoms. Significant blockage of the arteries can cause a heart attack. It can be diagnosed by imaging of the myocardium under rest or pharmacologic stress conditions to evaluate regional myocardial perfusion.

As used herein, the term ‘radionuclide or radioisotope’ refers to an unstable form of a chemical element that releases radiation as it breaks down and becomes more stable. Radionuclides can occur in nature or can be generated in a laboratory. In medicine, they are used in imaging tests and/or in treatment.

As used herein, the term ‘exponential function or multi-exponential function or mathematical function or square function based dosing’ refers to radionuclide dose calculation based on subject body habitus and/or other parameters as an exponential function. The parameters can include but are not limited to body weight, height, body mass index, body surface area, past medical history including medications, heart function including left ventricular and/or right ventricular ejection fraction, generator age, activity, infusion profile, infusion time, infusion mode. One example of exponential dosing can be calculated by equation: activity=0.1×weight², where weight is in kg and activity is in MBq. The exponential dosing protocol for Rb-82 was easy to implement clinically by the PET technologists as a simple calculation, i.e. activity=weight (kg)×weight (kg)±10. For example, an 85 kg patient would be prescribed the Rb-82 dose of 85×8.5=722.5 MBq (19.5 mCi). Patients of 149 kg would be given the maximum dose of 2220 MBq (60 mCi) as disclosed in US FDA approved label of RUBY-FILL®, manufactured by Jubilant DraxImage Inc. or 3700 MBq (100 mCi) for a 193 kg (425 lbs) patient as disclosed in the Health Canada approved PIL of RUBY-FILL®, manufactured by Jubilant DraxImage Inc. The activity available from the Rb-82 generator decreases over time according to the half-life of the parent 82Sr, from 3700 MBq on day 0 to 700 MBq on day 60. Therefore, to implement exponential Rb-82 dosing in practice, patient scheduling needs to be adjusted accordingly, with maximum patient weights up to 193 kg on day 0 and up to 84 kg on day 60. The present study results may be adapted to other PET perfusion imaging protocols, taking into account the differences in tracer retention fraction, isotope half-life, scan-time and PET scanner sensitivity. Rb-82 has approximately 30% tracer retention in the heart at a peak stress blood flow value of 3 mL/min/g, whereas other PET tracers such as 13N-ammonia or 18F-flurpiridaz have approximately 60% retention at peak stress, resulting in higher myocardial activity and image quality for the same injected dose. These longer half-life tracers typically require lower injected activity and scan-time that can be optimized for the desired image quality. These changes in imaging protocol should only affect the selected value of ε in equation 2, whereas the weight dependence of cardiac PET image quality (β) is expected to remain the same regardless of these tracer and protocol changes.

SNR_{Constant}=✓ε×Weight×k×Weight⁻¹=✓ε×k

According to one aspect of the present invention, the value of ε=0.1 MBq/kg² was selected to maintain the same Rb-82 image quality. This value is higher than those reported in prior art (0.023 to 0.053 MBq/kg²) to standardize 18 FDG PET image quality, likely due to the ultra-short half-life of Rb-82 resulting in much lower count-rate and image quality recorded per unit activity (MBq) injected.

As used herein, the term “Sr/Rb elution system” or “⁸²Sr/⁸²Rb elution system” refers to infusion system meant for generating a solution containing Rb-82, measuring the radioactivity in the solution, and infusing the solution into a subject in order to perform various studies on the subject region of interest.

As used herein, the terms image signal-to-noise ratio (SNR), contrast-to-noise ratio (CNR), image count, and coefficient of variation (COV) represent measures of image quality.

As used herein, the term “SNR” refers to signal to noise ratio, which is a measure of image quality. SNR can be defined as a ratio of target signal strength to the noise signal strength. Image quality is measured using signal to noise ratio or contrast to noise ratio in the heart walls compared to blood, lungs, liver, mediastinum or other reference organ or tissue.

As used herein, the term “CNR” refers to contrast to noise ratio, which is also a measure of image quality. CNR can be defined as a difference of target signal strength minus the background signal strength, divided by the noise signal strength.

As used herein, the term “image counts” refers to number of radioisotope disintegrations acquired per unit time by the PET scanner.

As used herein, the term “COV” refers to coefficient of variance/variation, which is a measure of background noise signal to define image quality. The value of calculated COV is used for calculation of SNR and CNR.

As used herein, the term “Image Quality Score (IQS)” refers to measure the image quality in consistent with subjective ratings by computational models. The objective of measurement to evaluate the quality of gray-scale compressed images denoted as Image Quality Score (IQS). The evaluation result is rated into 5—level grading scale, 1 to 5 (Poor, Fair, Good, Very Good and Excellent), which is comparable to Mean Opinion Score (MOS). The objective of this paper is to provide defining method, definition, and reliability of IQS. The IQS model is separated into three steps. First, the gray-scale values of original and compressed images, which are justified by peak signal are normalized that is divided by peak signal. Second, each measurement calculates the distortion and maps it into scale (1 to 5) by least square function calculated by holding to subjective measurement's principles. Finally, each scale is weighted and summed for providing IQS. The said IQS method can be performed by using specific algorithms for imaging processing, which is based on artificial intelligence (AI), deep learning, machine learning, artificial neural network and/or combinations thereof.

As used herein, the term “Ordered Subset Expectation Maximization (OSEM)” refers to the method, which is used in reconstruction algorithm for positron emission tomography (PET) images. In OSEM, data are first divided into subsets and then analyzed repetitively during iterations. In mathematical optimization, the ordered subset expectation maximization (OSEM) method is an iterative method that is used in computed tomography. In medical imaging as disclosed herein the present invention, the OSEM method is used for positron emission tomography (PET, single photon emission computed tomography (SPECT), and X-ray computed tomography. The OSEM method is related to the expectation maximization (EM) method of statistics. It is also related to methods of filtered back projection.

As used herein, the term “generator” or “radioisotope generator” refers to a hollow column inside a radio-shielded container. The column is filled with an ion exchange resin and radioisotope loaded onto the resin.

Radionuclide generator according to the present invention is selected from ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga ⁴²Ar/⁴²K, ⁴⁴Ti/⁴⁴Sc, ⁵²Fe/^{52m}Mn, ⁷²Se/⁷²As, ⁸³Rb/^{83m}Kr; ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Ir, ²²⁶Ra/²²²Rn and ²²⁵Ac/²¹³Bi.

As used herein, the term “eluant” refers to the liquid or the fluid used for selectively leaching out the daughter radioisotopes from the generator column.

As used herein, the term “eluate” refers to the radioactive eluant after acquisition of daughter radioisotope from the generator column.

As used herein, the term “controller” refers to a computer or a part thereof programmed to perform certain calculations, execute instructions, and control various activities of an elution system based on user input or automatically.

As used herein, the term “activity detector” refers to a component that is used to determine the amount of radioactivity present in eluate from a generator, e.g., prior to the administration of the eluate to the patient.

The present disclosure provides methods that result in improved image quality during radio-diagnosis procedures irrespective of subject body habitus variation.

In an embodiment according to the present invention, a novel method of PET or SPECT imaging is provided, wherein the dose is exponential function based on subject body habitus.

In an embodiment according to the present invention, consistent left ventricle (LV) myocardium signal-to-noise ratio (SNR) and myocardium-to-blood contrast-to-noise ratio (CNR) values across a wide range of patient body sizes when using exponential dosing of Rb-82 in comparison to a matched group of patients with linear dosing or fixed dosing is provided. The SNRₕₑₐᵣₜ and CNRₕₑₐᵣₜ values are in the range of 0-350 following proportional and exponential dosing in static and gated images quality.

In an embodiment according to the present invention, accurate Rb-82 injected activity over the full range of injected doses prescribed from 0.01 to 10,000 MBq is provided.

In an embodiment according to the present invention, a method of utilizing exponential function of the body habitus based dosing for PET imaging is provided for diagnosing a subject suffering from or at a risk of developing coronary artery disease, ischemic and non-ischemic heart disease, and other organ diseases such as liver, kidney, spleen, adrenal, pancreas, brain, inflammation related disorders like cancer, rheumatoid arthritis, infection, metabolic conditions like diabetes mellitus, thyroid malfunction and infections caused by pathogens like virus, bacteria and fungi or combinations thereof. By administration of proportional and exponential dosing of Rb-82 activity as disclosed herein, the present invention diagnoses the organ activity (heart) over a wide range of patient (smaller and larger) weights.

In an embodiment according to the present invention, a novel method of PET imaging is disclosed comprising administering a radionuclide to a subject, wherein the dose is based on exponential function of subject weight, body mass, height, age via automated generation and infusion system.

In an embodiment according to the present invention, the dose can be automatically calculated by automated generation and infusion system.

In an additional embodiment according to the present invention, automatic dose calculation further comprises other parameters selected from, type of radioisotope, radioisotope half-life, generator life (activity remaining in the radioisotope generator), generator yield, infusion time, flow rate, time lapse from generation to infusion of radioisotope, scanning instrument detector sensitivity, scanner resolution, type of camera or scanner, acquisition time, camera sensitivity, type of disease to be diagnosed, subject conditions like known allergies, heart function, liver function or kidney function or any other special need, subject's supplementary diseases, medications, type of imaging technique to be utilized like PET, SPECT, or combinations thereof.

In an embodiment according to the present invention, automated generation and infusion system comprises a cabinet, radioisotope generator, dose calibrator, computer, controller, display device, activity detector, cabinet, cart, waste bottle, sensors, shielding assembly, alarms or alerts mechanism, tubing, source vial, diluent or eluant, valves or combinations thereof. The automated generation and infusion system generates a radionuclide from a generator/column placed inside the system. A radionuclide eluate is generated from the generator by eluting the generator with suitable eluant like saline, which is then administered by the system automatically after activity measurements. The dose is calculated automatically by the system based on the entered subject parameters. The system is equipped to calculate the flow rate and infusion time depending on the dose to be administered. The automated generation and infusion system can comprise any radionuclide generator, which is suitable for administration to a subject like ⁸²Sr/⁸²Rb generator.

In an embodiment, the automated generation and infusion system is coupled to the imaging system electronically or communicatively. The coupled imaging system can provide error or alerts in case image quality is not up to the mark and require repeated administration or scanning.

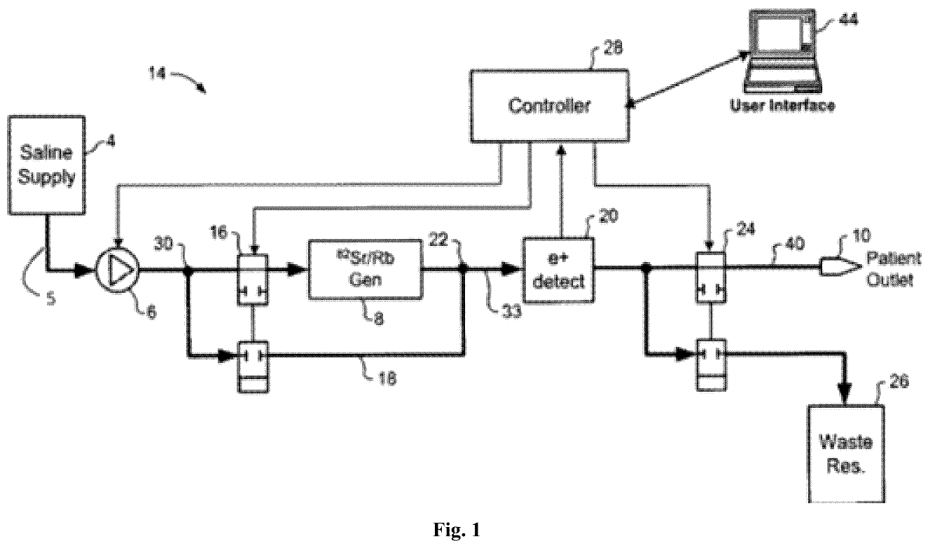

In an embodiment, the automated generation and infusion system is a rubidium (Rb-82) elution system, which comprises the components described in FIG. 1. In an embodiment, the elution system comprises reservoir 4 of sterile saline solution (e.g. 0.9% Sodium Chloride Injection); a pump 6 for drawing saline from the reservoir 4 through the supply line 5 and the generator line (between 30 and 22) at the desired flow rate; a generator valve 16 for proportioning the saline flow between a strontium-rubidium (⁸²Sr/⁸²Rb) generator 8 and a bypass line 18 which circumvents the generator 8; a positron detector 20 located downstream of the merge point 22 at which the generator and bypass flow merge; and a patient valve 24 for controlling supply of active saline to a patient outlet 10 and a waste reservoir 26. A controller 28 is preferably connected to the pump 6, positron detector 20 and valves 16 and 24 to control the elution system 14 in accordance with the desired control algorithm.

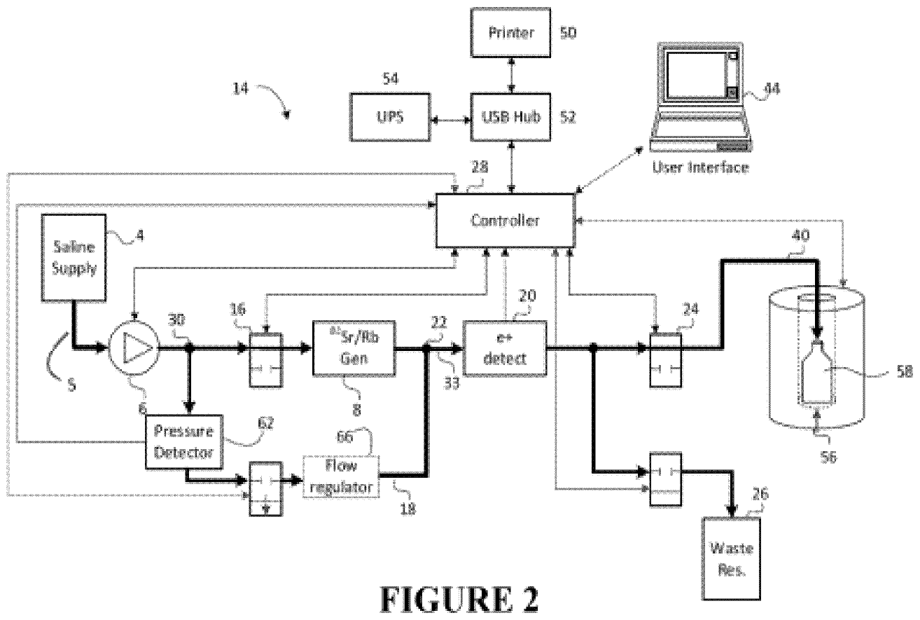

FIG. 2 Depicts a block diagram schematically illustrating principal elements of a rubidium elution system in accordance with another embodiment of the present invention. The rubidium elution system of FIG. 2 has similar elements as the Rubidium elution system of FIG. 1, and additional elements. These additional elements preferably include one or more of a printer 50 and USB (Universal Serial Bus; or other communications port) port 52, a pressure detector 62, a dose calibrator 56, a flow regulator 66, or a UPS (Uninterruptible Power Supply) 54.

The rubidium elution system of FIG. 2 can be used to assess various aspects of the system, such as a concentration of ⁸²Rb, ⁸²Sr, or ⁸⁵Sr in a fluid that is eluted from the generator, the volume of the fluid that is eluted from the generator, or the pressure of the fluid flowing through at least one portion of the system. Information about these aspects of the system can be gathered by various elements of the system and sent to the controller. The controller and/or user interface computer (which can comprise a processor and memory) can analyze this gathered data to assess the state of the system.

The rubidium elution system of FIG. 2 can additionally have a dose calibrator 56. The dose calibrator 56 can be used instead of a patient outlet, or in addition to a patient outlet, along with a valve that can be configured to direct fluid to the patient outlet or to the dose calibrator. The dose calibrator 56 can comprise a vial 58 (such as a 50 mL vial) that collects the fluid as it otherwise exits the elution system. The dose calibrator 56 can be electronically or communicatively coupled to the controller and configured to send information to the controller, such as an activity concentration of ⁸²Rb, ⁸²Sr, or ⁸⁵Sr in a fluid, which is eluted from the generator. The dose calibrator 56 can include a radioactivity shielding material.

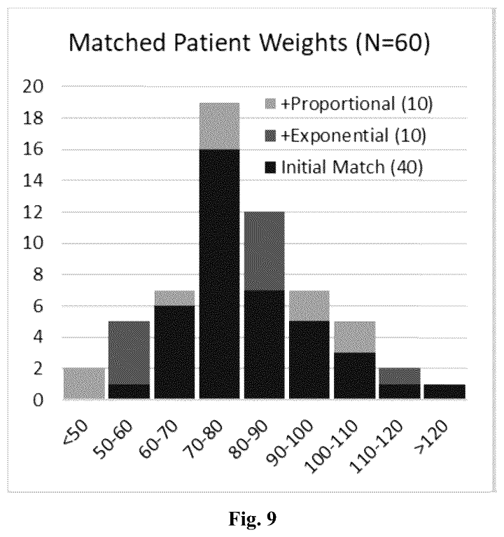

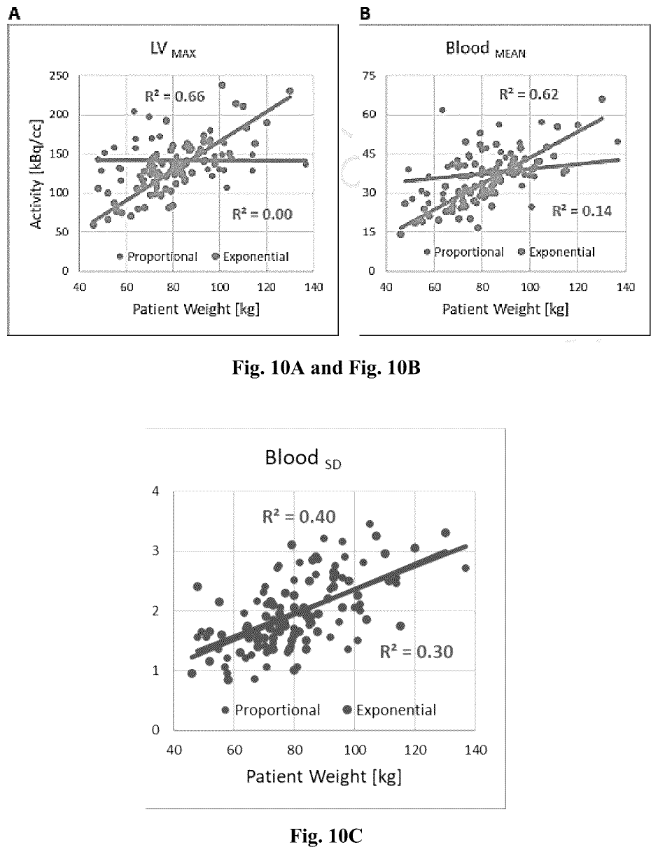

FIG. 9 demonstrates that the patient weight distributions in the exponential and proportional dosing cohorts were matched prospectively. FIG. 10 (A) represents Rb-82 PET activity values on ECG-gated imaging with proportional and exponential dosing. LV_{MAX} (A) values are constant with proportional dosing but increase linearly by weight with exponential dosing, (B) Blood_{MEAN} activity values are almost constant with proportional dosing but increase by weight with exponential dosing, and (C) Blood_{SD} activity remains very similar between dosing protocols.

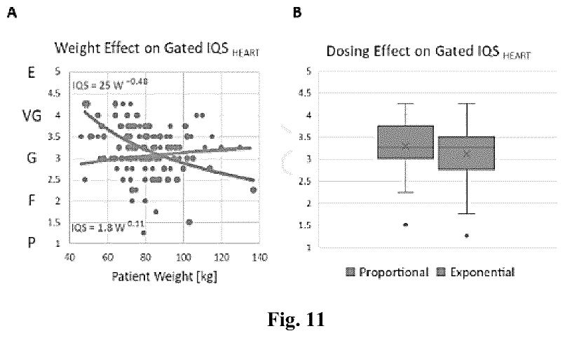

FIG. 11 demonstrates Rb-82 PET visual image quality score (IQS_{HEART}) was assessed on a 5-point scale (Excellent, Very Good, Good, Fair, Poor) which decreased by weight (A) in the proportional dosing group (orange) but was constant in the exponential dosing group (blue). There was no difference in the median ECG-gated image quality score (B) between dosing cohorts (P=0.11).

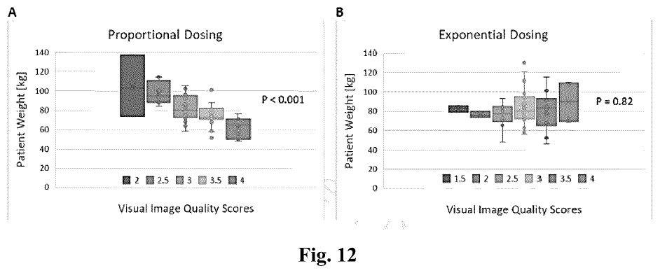

FIG. 12 demonstrates box-plots of patient weight according to visual image quality score (IQS) in the proportional (A) and exponential (B) dosing groups. There was a highly significant effect of increasing weight in patients with lower IQS in the proportion dosing group (P<0.001), whereas there was no such effect observed in the exponential dosing group (P=0.82) using Kruskal-Wallis tests.

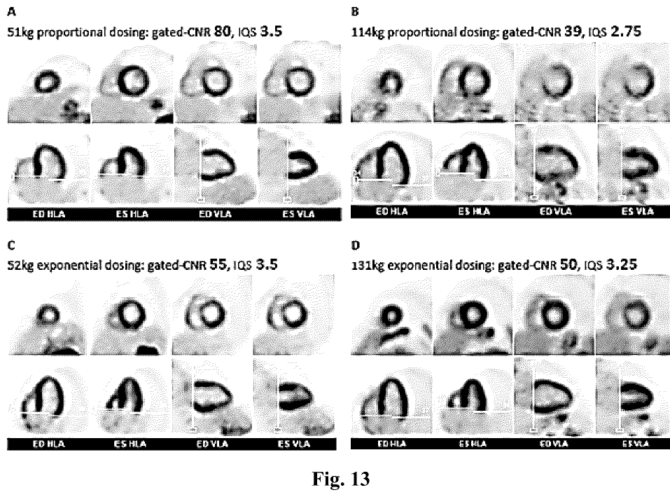

FIG. 13 demonstrates Rb-82 PET static-ungated SA (top) and ECG-gated HLA & VLA (bottom) images acquired with proportional (A, B) and exponential (C, D) dosing. Proportional dosing resulted in visibly lower image quality in the large (B) vs small (A) patient (CNR=39 vs 80). With exponential dosing the image quality was very similar between the large (D) and small (C) patient (CNR=50 vs 55), and much improved vs the large patient with proportional dosing (B).

FIG. 14 demonstrates Rb-82 PET static (ungated) images acquired with proportional (A, B) and exponential (C, D) dosing. Proportional dosing resulted in lower image quality in the large (B) vs small (A) patient (CNR=45 vs 200). With exponential dosing the image quality was more similar between the large (D) and small (C) patient (CNR=70 vs 120) and much improved vs the large patient with proportional dosing (B).

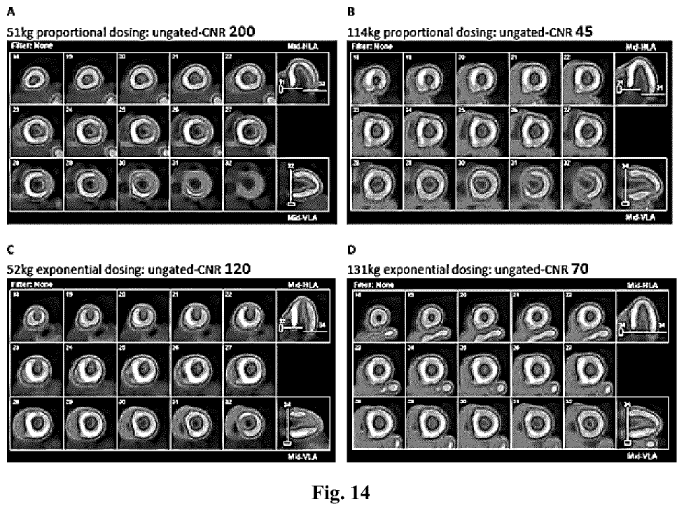

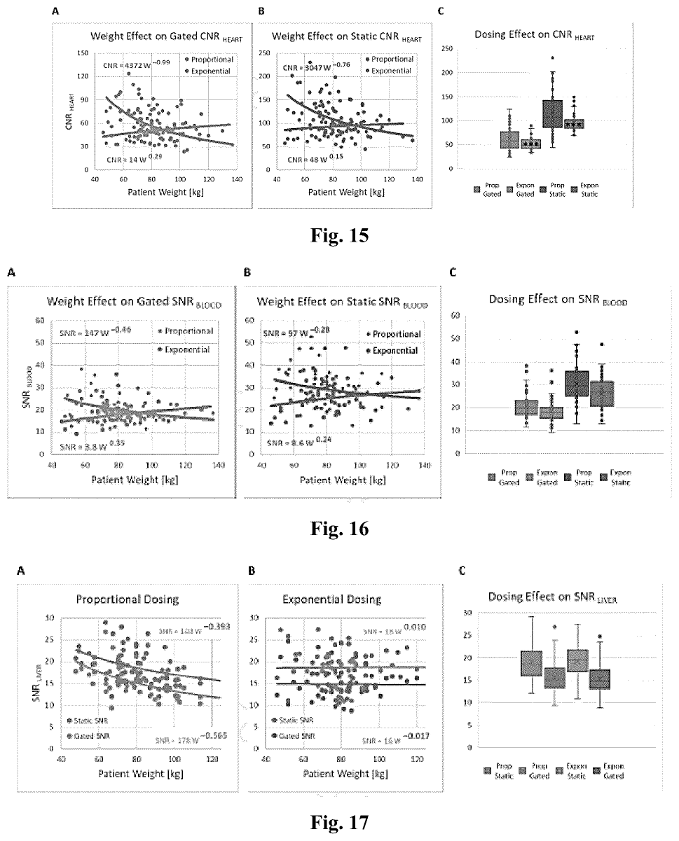

FIG. 15 demonstrates Rb-82 PET contrast-to-noise ratio (CNR_{HEART}) decreases with increasing patient body weight in the proportional dosing cohort but not in the exponential dosing cohort for both ECG-gated (A) and ungated static (B) images. (C) Box-plots of CNR_{HEART} in show there was a highly significant effect of exponential dosing to reduce the variability in image quality (CNR_{HEART}) among patients for both static and gated reconstructions (*** P<0.001 lower cohort variance versus proportional dosing).

FIG. 16 depicts Rb-82 PET signal-to-noise ratio (SNR_{BLOOD}) decreases with increasing patient body weight in the proportional dosing cohort (A) and tended to increase in the exponential dosing cohort (B). (C) Box-plots of the SNR_{BLOOD} show the summary effects of dosing method on the patient groups as a whole.

FIG. 17 demonstrates Rb-82 PET liver signal-to-noise ratio (SNR_{LIVER}) decreases with increasing patient body weight (W) in the proportional dosing cohort (A) but not in the exponential dosing cohort (B). (C) Box-plots of the SNR_{LIVER} show the summary effects of dosing method on the patient groups as a whole.

In an alternate embodiment according to the present invention, the automated generation and infusion system is embodied in a portable (or mobile) cart that houses some or all of the generator, the processor, the pump, the memory, the patient line, the bypass line, the positron detector, and/or the calibrator, sensors, dose calibrator, activity detector, waste bottle, controller, display, computer. The cart carrying the components for radioisotope generation and infusion is mobile and can be transferred from one place to another to the patient location or centers, or hospitals as required.

In another embodiment, the method of diagnosing/imaging blood perfusion or flow in the region of interest comprising: input subject parameters into the radioisotope generation and infusion system; automatically calculating the appropriate dose based on exponential function of subject body habitus; generating a radionuclide from automated generation or infusion system based on required dose to be administered; administering the radionuclide to the subject in need thereof; performing PET or SPECT scanning of the region of interest; automated analysis of the images by computerized software; quantitative assessment of the blood flow in the region of interest; generating automated report of the assessment; providing appropriate therapy options for the subject.

In an embodiment, the method of diagnosing/imaging a region of interest of a subject comprising: input one or more subject body habitus parameters into the rubidium elution system; automatically calculating the appropriate dose of Rb-82 based on one or more parameters; generating a dose of Rb-82 from rubidium elution system; administering Rb-82 to the subject in need thereof; performing PET scanning of the region of interest; automated analysis of the images by computerized software; quantitative assessment of the blood flow in the region of interest; generating automated report of the assessment; providing appropriate therapy options for the subject.

In another embodiment of the present invention, the method further comprises administration of a stress agent selected from adenosine, adenosine triphosphate, regadenoson, dipyridamole, and dobutamine.

In another embodiment of the present invention, signal to noise ratio ranges from 1 to 1000 dB (decibel).

In another embodiment of the present invention, contrast to noise ratio ranges from 1 to 1000 dB (decibel).

In another embodiment, the image quality is measured by determination of coefficient of variation in the image quality represented by signal to noise ratio or contrast to noise ratio when dosing is exponential function of weight in comparison to linear dosing based on subject weight in the range of 1 kg to 300 kg. The consistency of image quality is represented by coefficient of variation. Coefficient of variation value can be expressed as percentage variation for signal to noise ratio in exponential function of weight-based dosing. Coefficient of variation for exponential weight based dosing ranges from about 15 to 30 percent in comparison to linear based dosing having coefficient of variation of more than 30 percent.

In an embodiment, the subject weight is in the range of 1 kg to 300 kg, preferably in the range of 2 kg to 190 kg.

In another embodiment, the method comprises providing treatment options to a subject based on the severity of the disease.

In another aspect of the present invention, the method comprises monitoring of the disease during treatment.

In yet another embodiment, the present invention relates to a method of imaging a subject suffering from or at a risk of developing a coronary artery disease comprising: calculating the dose based on one or more parameters selected from subject parameters, infusion system parameters, imaging system parameters or combinations thereof; generating a dose of radioisotope by automated radioisotope generation and infusion system; administering the dose of generated radioisotope to the subject; performing PET or SPECT imaging to obtain images; administering the dose of stress agent and performing PET or SPECT imaging to obtain images; performing an assessment of the obtained images.

In an embodiment, radioisotope can be generated by automated generation or infusion system or can be generated at a remote location like radioisotope generation facility or radiopharmacy or other centers in bulk and then placed in the radioisotope generation and infusion system for dilution and/or administration to the patient automatically.

In one embodiment, the radionuclide can be attached to the ligand before administration into the subject. The ligands are provided in a suitable dosage form and radionuclide is attached to the ligand and then administered to the subject for imaging.

In an embodiment, the subject is a human subject. The human subject is a male or female subject. The age of the subject may vary from 1 month to 120 years. The human subject includes neonate, pediatric, adult and/or geriatric population.

In the present application, all numbers disclosed herein can vary by 1 percent, 2 percent, 5 percent, or up to 20 percent if the word “about” is used in connection therewith. This variation may be applied to all numbers disclosed herein.

Each embodiment disclosed herein is contemplated as being applicable to each of the other disclosed embodiments. Thus, all combinations of the various elements described herein are within the scope of the invention.

This invention will be better understood by reference to the experimental data, which follow, but those skilled in the art will readily appreciate that the specific experiments detailed are only illustrative of the invention as described more fully in the claims, which follow thereafter.

### EXPERIMENTAL

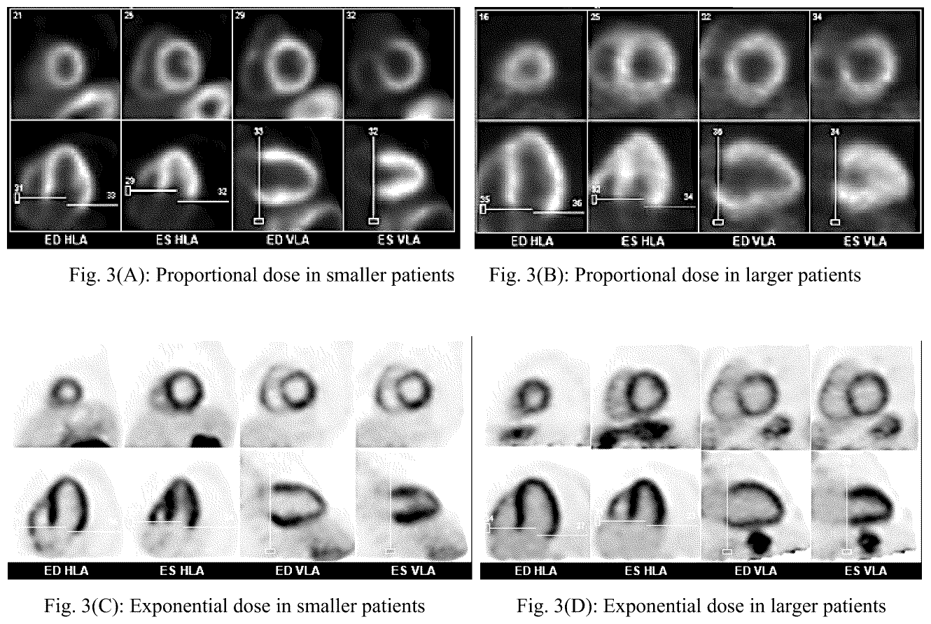

Dosing based on linear function of subject weight resulted in poor image quality, especially in larger subjects. Rb-82 PET images in a 35 kg patient and a 180 kg patient were acquired following linear weight-based administration of approximately 5-20 MBq/kg tracer activity. Lower image quality was observed in the larger patient (FIG. 3). To overcome this undesirable limitation of weight based dosing as a linear function of patient weight or fixed dosing, the inventors of the present invention propose dosing based on exponential of subject body habitus, as described herein.

The study described herein was an interrupted time series cohort comparison study. An exponential dosing protocol was designed to increase the ⁸²Rb activity as a squared function of body weight, while maintaining the same injected activity as the previous proportional dosing function for patients with a population average weight of 90 kg.

A control group of 50 consecutive patients underwent clinically indicated ⁸²Rb MPI imaging with proportional dosing (9 MBq/kg) during a 2-week period. Following a short transition period, an additional 50 consecutive patients underwent clinically indicated ⁸²Rb myocardial perfusion imaging (MPI) with the exponential dosing protocol (0.1 MBq/kg²) during a 1-week period. The distribution of patient weights was compared between cohorts in 10 kg intervals. In those intervals with unequal numbers, subsequent consecutive patients in each cohort (N=10) were added to obtain a matched weight distribution consisting of N=60 patients in both groups. The demographics of the patient population is provided in Table 1.

TABLE 1
Demographics of the Patient Population

| | Proportional | Exponential | |
|---|---|---|---|
| | Dosing | Dosing | |
| Description | (N = 60) | (N = 60) | P-value |
| Age (years) | 65 ± 14 | 69 ± 11 | 0.086 |
| Female sex | 27 (45%) | 28 (47%) | 0.855 |
| Weight (kg) | 81 ± 18 | 81 ± 18 | 0.960 |
| Body Mass Index (kg/m²) | 29 ± 7.5 | 29 ± 6.2 | 0.919 |
| Coronary Risk Factors | | | |
| Hypertension | 39 (65%) | 41 (68%) | 0.697 |
| Dyslipidemia | 43 (72%) | 45 (75%) | 0.682 |
| Family history | 28 (47%) | 26 (43%) | 0.711 |
| Smoking (current or past) | 35 (58%) | 36 (60%) | 0.849 |
| Diabetes (type I or II) | 15 (25%) | 14 (23%) | 0.834 |
| Angina symptoms | | | |
| None | 35 (58%) | 28 (47%) | 0.201 |
| Typical | 8 (13%) | 10 (17%) | 0.610 |
| Atypical | 5 (8%) | 10 (17%) | 0.168 |
| Non-anginal | 12 (20%) | 12 (20%) | 1.000 |
| Cardiac history | | | |
| Previous Myocardial Infarction | 10 (17%) | 19 (32%) | 0.055 |
| Previous Percutaneous | 11 (18%) | 12 (20%) | 0.818 |
| Intervention | 3 (5%) | 6 (10%) | 0.298 |
| Previous Coronary Bypass | | | |
| Grafting | | | |

Values are mean ± standard deviation or N (%)
No significant differences between dosing cohorts

Both proportional and exponential cohort scans were acquired on a Biograph Vision600 PET-CT scanner (Siemens Healthcare, Hoffman Estates, IL) following a standard clinical protocol. Briefly, a low-dose CT scan was performed at normal end-expiration for attenuation correction of the rest and stress PET scans. Dynamic PET imaging was started together with a 30-second square-wave injection of Rubidium Rb 82 Chloride injection (RUBY-FILL™, Jubilant DraxImage, QC) at rest and again during dipyridamole stress. Ungated static images were reconstructed from 2 to 8 minutes, and ECG-gated images from 1½ to 8 minutes following tracer injection to maximize count statistics following the blood clearance phase. The vendor iterative OSEM reconstruction method was used including time-of-flight with 5 subsets, 4 iterations and 6 mm Gaussian post-filtering.

### Results

The proportional and exponential dosing cohorts had similar clinical characteristics, including patient weights as expected based on the prospective cohort matching (FIG. 9). The median injected activity was 12% lower using exponential vs proportional dosing (p=0.04), as the median weight in our experimental cohort (80 kg) was slightly lower than the historical value of 90 kg used to design the exponential dosing protocol. The min-max range was substantially wider (211-1850 vs 433-1362 MBq) as expected using exponential vs proportional dosing. With proportional dosing the mean activity values in the LV myocardium and blood were relatively constant whereas with exponential dosing they both increased linearly with patient body weight (FIG. 10A, 10B). Background noise (SNR_{BLOOD}) in both cohorts increased linearly with body weight and was unchanged between dosing protocols (FIG. 10C).

For the measurements of cardiac IQS, CNR and SNR, the inter-operator agreement was excellent with mean differences≤5% (Table 3). The average values of IQS, CNR and SNR are shown for both dosing cohorts in Table 2. In the exponential dosing cohort, there was an average decrease of −8.5% across all image quality metrics, consistent with the lower average injected activity as noted earlier. More importantly, there was 40% decreased variability of both the static and gated CNR_{HEART} values in the exponential dosing cohort (P<0.001) demonstrating significantly improved consistency of image quality compared to proportional dosing.

TABLE 2
82Rb PET image quality measurements

| Image Quality | | Proportional | Exponential |
|---|---|---|---|
| IQS_{HEART} | Gated | 3.1 ± 0.6 | 3.3 ± 0.5 |
| CNR_{HEART} | Static | 117 ± 45 | 95 ± 27* |
| | Gated | 61 ± 23 | 51 ± 14* |
| SNR_{BLOOD} | Static | 30 ± 8 | 27 ± 6 |
| | Gated | 21 ± 5 | 18 ± 5 |
| SNR_{LIVER} | Static | 19 ± 3.8 | 19 ± 4.2 |
| | Gated | 16 ± 3.5 | 15 ± 3.6 |

IQS is Image Quality Score, SNR is Signal-to-Noise Ratio, CNR is contrast-to-Noise Ratio Values are mean ± standard deviation
*P < 0.001 lower variance versus proportional dosing cohort

TABLE 3
Operator reproducibility of image quality

| Dosing Cohort | IQS_{HEART} | CNR_{HEART} | SNR_{BLOOD} |
|---|---|---|---|
| Static Proportional | — | 4 ± 37% | 5 ± 35% |
| Gated Proportional | −2 ± 14% | 2 ± 34% | 1 ± 32% |
| Static Exponential | — | −3 ± 34% | −1 ± 32% |
| Gated Exponential | −2 ± 15% | 0 ± 32% | 0 ± 27% |

Values are mean difference ± standard deviation between operators No significant differences versus zero IQS (Imaging quality score), CNR (Contrast-to-noise ratio), SNR (Signal-to-noise ratio)

Improved consistency was confirmed with the visual image quality scores (FIG. 11) in the exponential dosing cohort, which showed no significant dependence on body weight ((3=P=0.38). This was in contrast to the proportional dosing group which showed a significant decrease in image quality ((3=−0.48; P<0.001) that was very similar to the value predicted by Equation 1:

SNR_{Target}=✓At×k×Weight^{β} (1)

Interestingly, the crossing point of equivalent IQS_{HEART} values in both cohorts was close to 90 kg, further demonstrating validity of the noise model and dosing methods as described in the embodiments of the present invention. Higher body weight was observed in the patients with lower IQS in the proportional dosing cohort (P<0.001) but with not exponential dosing (P=0.82) where the distribution of weights was uniform across different visual IQS values (FIG. 12). The changes in visual image quality between dosing methods can be seen in the patient examples shown in FIGS. 13 (A, B, C and D) and FIG. 14.

### Analysis of PET Image Quality

ECG-gated and ungated (static) PET images were analyzed at stress from two cohorts referred for Rb-82 MPI on a Siemens Vision 600 PET-CT scanner with approximately 200 ps time-of-flight (TOF) resolution. Ungated static and ECG gated images were set with the time duration following tracer injection to maximize count statistics in the blood clearance phase. The OSEM reconstruction method was used with specified Gaussian filters. Myocardium signal recovery was measured as the maximum activity in the left ventricle (LV_{MAX}) at end-diastole (ED). Corresponding background signal and noise were measured as the left atrium blood cavity mean and standard deviation (BL_{MEAN} and BL_{SD}). Myocardium signal-to-noise ratio (SNR=LV_{MAX}/BL_{SD}) and myocardium-to-blood contrast-to-noise ratio (CNR=(LV_{MAX}−BL_{MEAN})/BL_{SD}) were calculated for both the static and ECG-gated end-diastolic images.

### Statistical Analysis

Two operators performed PET image analysis as described above. Measurements of LV_{MAX}, BL_{MEAN}, BL_{SD}, SNR and CNR were compared between operators using Bland-Altman and Box-plot analyses. The mean values between operators were used in the final analysis of weight-based effects. SNR and CNR were fit to power functions of patient weight Beta in both patient groups, and Beta coefficients were compared between the exponential and linear dosing groups using 95% confidence intervals. It is recommended to determine beta coefficient values (SNRₕₑₐᵣₜ and CNRₕₑₐᵣₜ) in the range of −1.5 to 1. SNR_{LV} would be recommended in subjects without CAD to ensure homogeneous tracer uptake. Variances were compared using Levene's tests. Mean values were compared using paired Student t-tests, and median values using Mann-Whitney U tests. P<0.05 was considered statistically significant. Statistical analysis was performed using Excel 2019 with Real Statistics 8.1.

### Results

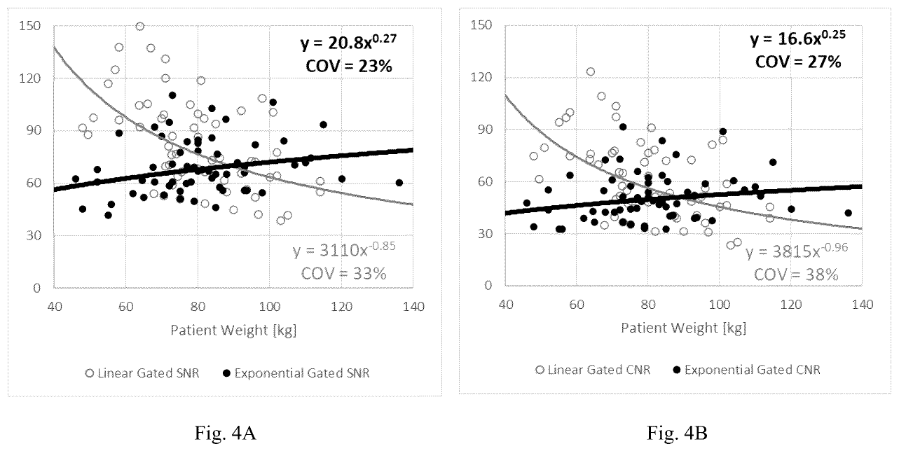

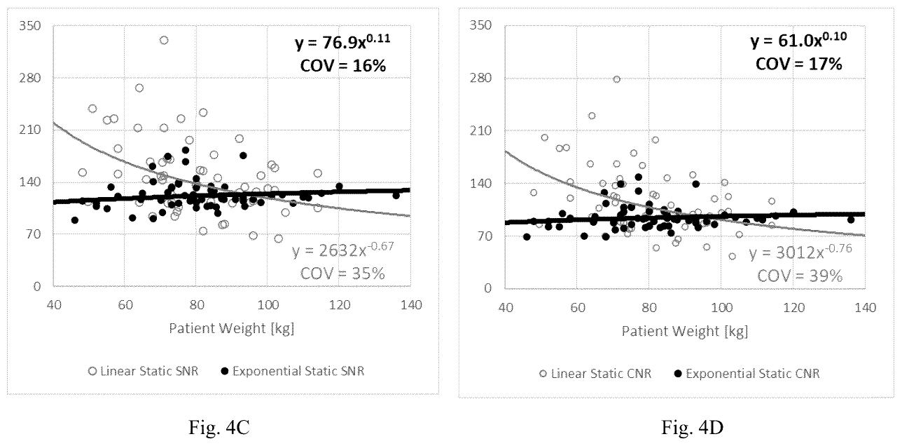

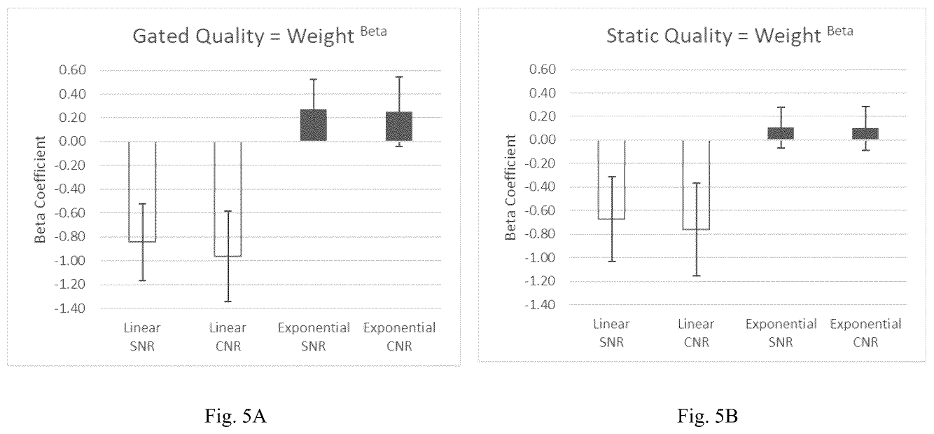

The linear and exponential dosing cohorts had the same mean and variance of patient weights 81±18 kg. The signal to noise ratio and contrast to noise ratio data are shown in FIG. 4, clearly demonstrating better uniformity of image quality in the exponential dosing group in comparison to fixed and linear dosing. Image quality (SNR) was expected to change as a function of weight⁻¹ with linear dosing, and improve to weight⁰ (no weight-dependence) with exponential dosing. The measured power function exponent values (FIG. 5) show that image quality is no longer a significant function of weight in the exponential dosing cohort (95% CI including zero), whereas it was in the linear dosing cohort. The average exponent values are also close to the expected values: −0.8 (linear) and +0.2 (exponential) and the difference (+1.0) is exactly equal to the expected improvement in image quality. PET image quality is determined by count statistics which follow a Poisson distribution, first order and De Groot analysis. As depicted in FIG. 4, the coefficient of variation for exponential weight based dosing was found to be in the range of 16 to 27 percent for static and gated imaging and the coefficient of variation for linear weight based dosing was found in the range of 33 to 39 percent for static and gated imaging scans.

The quantitative CNR_{HEART} values shown in FIG. 7 demonstrated even more pronounced effects compared to the visual IQS_{HEART} scores. Both the ECG-gated and static images had better consistency of image quality in the exponential vs proportional dosing group (FIGS. 15A and 15B). Proportional dosing resulted in significantly decreased CNR_{HEART} with increasing weight ((3=−0.99 and −0.76, both P<0.001), whereas there was no significant weight effect in the exponential dosing cohort ((3=0.29 and 0.08, both P>0.05). The corresponding effects of dosing protocol on SNR_{HEART} and SNR_{LIVER} were also very similar, as shown in the FIG. 16 and FIG. 17.

The β coefficients summarizing the weight-dependence of all the image quality metrics are shown in Table 3. In the proportional dosing cohort, the average coefficient was (β=−0.56) confirming the negative effect of patient weight on image quality that was predicted in FIG. 1B. In the exponential dosing cohort, the average coefficient was (β=0.19) suggesting a possible small effect to actually increase quality in the gated and static images of the larger patients. The result of the present invention suggests that an exponential dosing coefficient slightly less than the squared function that is evaluated (exponent<2) may have been sufficient to remove the weight-dependence of image quality. On the other hand, the squared function did produce very consistent results between visual IQS and quantitative CNR_{HEART} which were both based on the combined evaluation of myocardium to blood contrast and background noise.

TABLE 3
Weight-dependence of Rb-82 PET image quality

| | Proportional | Exponential | Exponential- |
|---|---|---|---|
| β Coefficients | Dosing | Dosing | Proportional |
| Gated IQS_{HEART} | −0.48* | +0.11 | +0.59 |
| Static CNR_{HEART} | −0.76* | +0.15 | +0.91 |
| Gated CNR_{HEART} | −0.99* | +0.29 | +1.28 |
| Static SNR_{BLOOD} | −0.28 | +0.24 | +0.52 |
| Gated SNR_{BLOOD} | −0.46* | +0.35* | +0.81 |
| AVERAGE_{HEART} | −0.59 | +0.23 | +0.82 |
| Static SNR_{LIVER} | −0.39* | +0.01 | +0.40 |
| Gated SNR_{LIVER} | −0.56* | −0.02 | +0.54 |
| AVERAGE_{LIVER} | −0.48 | −0.01 | +0.47 |

*P < 0.05 compared to zero

### Discussion

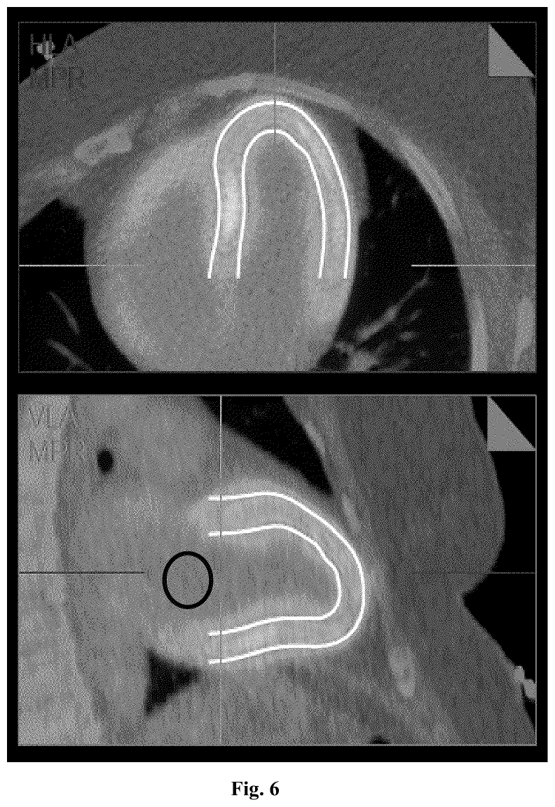

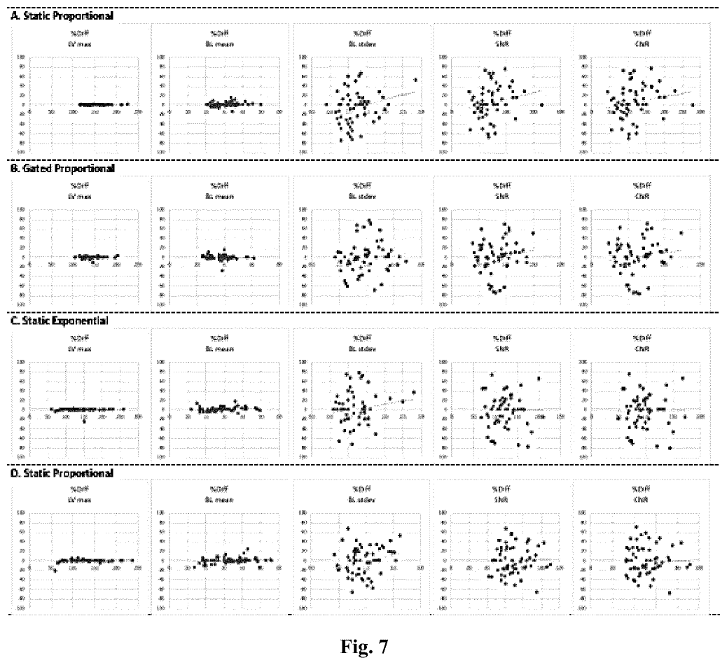

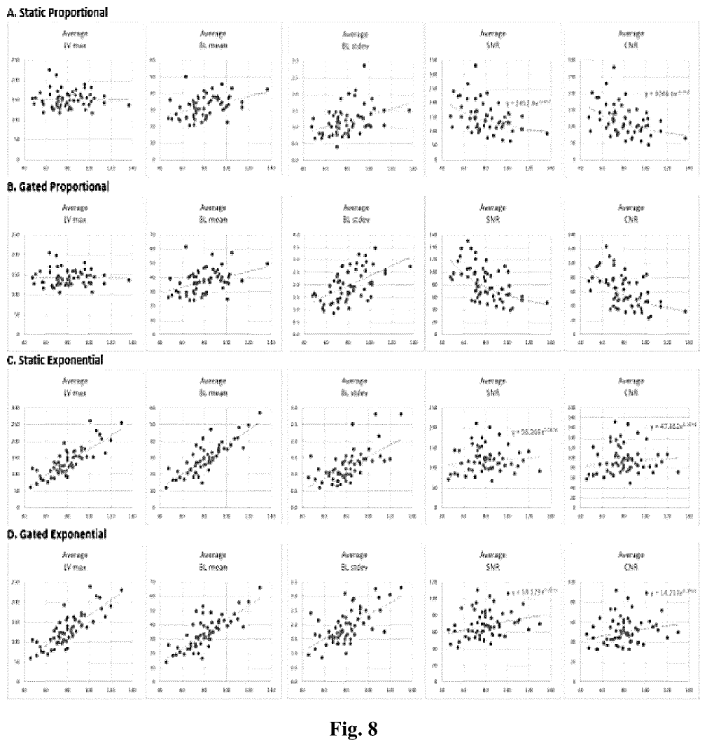

The inventor believes this to be the first report of a patient-centered approach using exponential dosing to standardize image quality for ⁸²Rb PET perfusion imaging. In the control group, when ⁸²Rb activity was administered in proportion to patient weight (9 MBq/kg) image quality was observed to decrease significantly with increasing body weight (β values<0). For each 10 kg increase in patient weight, the ECG-gated CNR decreased by approximately 10%. This is equivalent to 50% reduction in CNR when the patient weight is doubled from 50 kg (110 lbs) to 100 kg (220 lbs), similar to the reduction shown in the patient examples of FIGS. 6A, 6B. Conversely, in the experimental group using exponential dosing (0.1 MBq/kg2) the image quality was more consistent (β values≈0) with less than 10% variation on average across a wide range of patient weights ranging from approximately 50 kg to 120 kg. The biggest changes in activity occurred at the extremes of patient weight, essentially redistributing the population dose from the smaller to the larger patients as needed to standardize image quality.

The inventors have shown that using an exponential dosing based on a body habitus measure, image quality is unexpectedly improved compared to the image quality resulting from proportional dosing based on the same body habitus. Although body weight was used as the body habitus measure in the study reported above, other body habitus measures can be used, including body height, body surface area, lean body mass, body mass index, thoracic, and abdominal circumference and combinations thereof (including with body weight). Thus, in summary, the invention includes a process of imaging by (1) measuring or determining a body habitus, (2) calculating a dose of Rb-82 based on the exponential function of body habitus (e.g., the square of body habitus), (3) generating the calculated dose of Rb-82 by an automated elution system, (4) administering the generated dose of Rb-82 to the subject with the measured body habitus, (5) performing PET imaging on the subject, and (6) performing an assessment of the obtained images to diagnose a disease state. The process of imaging is preferably applied to coronary artery disease imaging. The invention also includes the steps of (1) measuring or determining a body habitus, and (2) calculating a dose of Rb-82 based on the exponential function of body habitus (e.g., the square of body habitus).

What is claimed:

1. A method of imaging processing for diagnosing and/or identifying a risk of developing a coronary artery disease comprising administering a dose of Rb-82 to a subject, wherein the dose is calculated based on exponential squared function of body habitus of the subject; and wherein the method of imaging processing in a subject is iterative ordered-subset expectation maximisation (OSEM) reconstruction method.

2. The method according to claim 1, wherein the body habitus comprises body weight, body height body surface area, lean body mass, body mass index, and thoracic or abdominal circumference or combinations thereof.

3. The method according to claim 1, wherein the dose can be further adjusted based on additional parameters selected from the group consisting of left ventricle ejection fraction, infusion time, infusion rate, imaging scanner sensitivity, type of radionuclide, imaging scanner/camera resolution and radionuclide generator age, generator yield or combination thereof.

4. The method according to claim 1, wherein the method of imaging processing is based on artificial intelligence (AI), deep learning, machine learning, artificial neural network and/or combinations thereof.

5. The method according to claim 1, wherein the iterative ordered-subset expectation maximization (OSEM) reconstruction method is based on time-of-flight (TOF) model.

6. The method according to claim 5, wherein the time-of-flight (TOF) model includes 5 subsets, 4 iterations, 128 matrix size with 4×4×3 mm voxels.

7. The method according to claim 5, wherein the time-of-flight (TOF) model includes 6 mm gaussian post-filtering.

8. The method according to claim 1, wherein the imaging agent or radionuclide is administered by automated generation and infusion system.

9. The method according to claim 8, wherein automated radioisotope generation and infusion system comprises Rb-82 elution system.

10. The method according to claim 1, wherein the dose is based on exponential function of the subject weight.

11. The method according to claim 1, wherein exponential squared function based dosing is calculated by activity is equal to 0.1×weight², wherein the weight is in kilograms and activity is in MBq.

12. The method according to claim 1, wherein consistent image quality is observed in the dose range of 1 MBq to 10,000 MBq and wherein the subject weight ranges from 1 kg to 300 kg.

13. The method according to claim 1, wherein the method further comprises administering a stress agent to the subject and wherein the stress agent is selected from the group consisting of adenosine, adenosine triphosphate, regadenoson, dobutamine, dipyridamole, exercise and/or combinations thereof.

14. A method of obtaining Rb-82 positron emission tomography images of a region of interest of a subject having consistent image quality, wherein the dose of imaging agent is calculated based on exponential squared function of the subject's body habitus.

15. The method according to claim 14, wherein the image quality is independent of body habitus variation in the subjects.

16. The method according to claim 14, wherein the consistency of image quality is measured by coefficient of variation of signal to noise ratio and/or contrast to noise ratio measured over a subject weight range of 10 kg to 200 kg for exponential weight based dosing and linear weight based dosing.

17. A method of obtaining Rb-82 positron emission tomography images of a region of interest of a subject having consistent image quality, wherein the dose of imaging agent is calculated based on exponential squared function of body habitus of the subject; and wherein the method of imaging the subject is iterative ordered-subset expectation maximization (OSEM) reconstruction method.

18. The method according to claim 1, wherein the method is used to measure the visual image quality scoring (IQS) of the region of interest of the subject.

19. The method according to claim 1, wherein the method of imaging is selected from the group consisting of positron emission tomography imaging (PET), dynamic positron emission tomography imaging (dynamic-PET), single-photon emission computed tomography (SPECT) imaging and/or combinations thereof.

20. A method of imaging a subject suffering from or at a risk of developing a coronary artery disease comprising:
a) calculating a dose of Rb-82 based on exponential squared function of body habitus of the subject;
b) generating a calculated dose of Rb-82 by automated elution system;
c) administering the generated dose of Rb-82 to the subject;
d) performing positron emission tomography imaging to obtain images; and
e) performing an assessment of the obtained images to diagnose disease state;
wherein the method of imaging the subject is iterative ordered-subset expectation maximization (OSEM) reconstruction method in order to exhibit qualitative visual image quality scoring (IQS) and quantitative contrast-to-noise ratio (CNR) and blood background signal-to-noise ratio (SNR) as a function of body weight.
